(19) United States
(12) Patent Application Publication
(10) Pub. No.: US 2011/0212894 A1
(43) Pub. Date: Sep. 1, 2011

(54) DECELLULARIZATION METHOD FOR SCAFFOLDLESS TISSUE ENGINEERED ARTICULAR CARTILAGE OR NATIVE CARTILAGE TISSUE

(76) Inventors: Kyriacos A. Athanasiou, Davis, CA (US); Benjamin Daniel Elder, Perry Hall, MD (US)

(21) Appl. No.: 13/029,325

(22) Filed: Feb. 17, 2011

Related U.S. Application Data
(63) Continuation of application No. PCT/US2009/054191, filed on Aug. 19, 2009.
Application No. PCT/US2009/054191 is a continuation-in-part of application No. 12/874,803, filed on Sep. 2, 2010.
Application No. 12/874,803 is a continuation of application No. PCT/US2009/035712, filed on Mar. 2, 2009.
Application No. PCT/US2009/035712 is a continuation-in-part of application No. 12/246,306, filed on Oct. 6, 2008.
Application No. 12/246,306 is a continuation-in-part of application No. PCT/US07/66092, filed on Apr. 5, 2007.
Application No. 12/246,306 is a continuation-in-part of application No. PCT/US2007/066089, filed on Apr. 5, 2007.
Application No. 12/246,306 is a continuation-in-part of application No. PCT/US2007/066085, filed on Apr. 5, 2007.
Application No. PCT/US2007/066085 is a continuation-in-part of application No. 12/246,320, filed on Oct. 6, 2008.
Application No. 12/246,320 is a continuation-in-part of application No. PCT/US07/66092, filed on Apr. 5, 2007.
Application No. 12/246,320 is a continuation-in-part of application No. PCT/US07/66089, filed on Apr. 5, 2007.
Application No. 12/246,320 is a continuation-in-part of application No. PCT/US07/66085, filed on Apr. 5, 2007.
Application No. PCT/US07/66085 is a continuation-in-part of application No. 12/246,367, filed on Oct. 6, 2008.
Application No. 12/246,367 is a continuation-in-part of application No. PCT/US2007/066092, filed on Apr. 5, 2007.
Application No. 12/246,367 is a continuation-in-part of application No. PCT/US2007/066089, filed on Apr. 5, 2007.
Application No. 12/246,367 is a continuation-in-part of application No. PCT/US2007/066085, filed on Apr. 5, 2007.
Application No. PCT/US2007/066085 is a continuation-in-part of application No. 11/571,790, filed on Aug. 23, 2010.
(60) Provisional application No. 61/033,094, filed on Mar. 3, 2008.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/789,855, filed on Apr. 5, 2006.
(60) Provisional application No. 60/789,853, filed on Apr. 5, 2006.
(60) Provisional application No. 60/789,851, filed on Apr. 5, 2006.
(60) Provisional application No. 60/586,862, filed on Jul. 9, 2004.

(51) Int. Cl. A61K 38/39; C12P 21/00; A61P 43/00

(52) U.S. Cl. 514/17.2; 435/70.3

(57) ABSTRACT

Methods for fabricating a tissue-engineered construct comprising: providing a tissue-engineered construct, wherein the tissue-engineered construct is derived from a xenogenic source; and decellularizing the tissue-engineered construct.

### CROSS-REFERENCE TO RELATED APPLICATIONS

This application is a continuation of International Patent Application No. PCT/US2009/54191 filed Aug. 19, 2009, which claims the benefit of U.S. Provisional Application No. 61/089,703, filed Aug. 18, 2008, the entire disclosures of which are incorporated by reference; a continuation-in-part of U.S. patent application Ser. No. 12/874,803, filed Sep. 2, 2010, which is a continuation of International Application No. PCT/US2009/035712, filed Mar. 2, 2009, which claims the benefit of U.S. Provisional Patent Application Ser. No. 61/033,094, filed Mar. 3, 2008, the entire disclosures of which are incorporated by reference; a continuation-in-part of U.S. patent application Ser. No. 12/246,306 filed Oct. 6, 2008, which is a continuation-in-part of International Patent Application Nos. PCT/US07/066,092 filed Apr. 5, 2007, PCT/US2007/066089 filed Apr. 5, 2007, and PCT/US2007/066085 filed Apr. 5, 2007, all of which claims the benefit of U.S. Provisional Application Nos. 60/789,855 filed Apr. 5, 2006, 60/789,853 filed Apr. 5, 2006, and 60/789,851 filed Apr. 5, 2006, the entire disclosures of which are incorporated by reference; a continuation-in-part of U.S. patent application Ser. No. 12/246,320 filed Oct. 6, 2008, which is a continuation-in-part of International Patent Application Nos. PCT/US07/066,092 filed Apr. 5, 2007, PCT/US2007/066089 filed Apr. 5, 2007, and PCT/US2007/066085 filed Apr. 5, 2007, all of which claims the benefit of U.S. Provisional Application Nos. 60/789,855 filed Apr. 5, 2006, 60/789,853 filed Apr. 5, 2006, and 60/789,851 filed Apr. 5, 2006, the entire disclosures of which are incorporated by reference; a continuation-in-part of U.S. patent application Ser. No. 12/246,367 filed Oct. 6, 2008, which is a continuation-in-part of International Patent Application Nos. PCT/US07/066,092 filed Apr. 5, 2007, PCT/US2007/066089 filed Apr. 5, 2007, and PCT/US2007/066085 filed Apr. 5, 2007, all of which claims the benefit of U.S. Provisional Application Nos. 60/789,855 filed Apr. 5, 2006, 60/789,853 filed Apr. 5, 2006, and 60/789,851 filed Apr. 5, 2006, the entire disclosures of which are incorporated by reference; a continuation-in-part of U.S. U.S. patent application Ser. No. 11/571,790 filed Jan. 8, 2007, which claims the benefit of International Application No. PCT/US2005/24269 filed Jul. 8, 2005, which claims the benefit of U.S. Provisional Application No. 60/586,862 filed Jul. 9, 2004, the entire disclosures of which are incorporated by reference.

### BACKGROUND

The present invention relates generally to processes that eliminate cells from scaffold-free engineered constructs, yielding a non-immunogenic xenogenic product intended for tissue replacement.

Injuries to articular cartilage, whether traumatic or from degeneration, generally result in the formation of mechanically inferior fibrocartilage, due to the tissue's poor intrinsic healing response. As such, tissue engineering strategies have focused on developing replacement tissue in vitro for eventual in vivo implantation. One such strategy employs a “self-assembly process” in which chondrocytes can be used to form robust tissue engineered constructs without the use of a scaffold.

Although engineered articular cartilage tissue has recently been created with biochemical and biomechanical properties in the range of native tissue values, there are currently two significant limitations to cartilage tissue engineering. First, human cells are scarce in number and difficult to procure, and passage of these cells leads to dedifferentiation. These issues make the use of autologous cells for cartilage repair difficult. Additionally, the majority of cartilage tissue engineering approaches have employed bovine or other animal cells, and tissues grown from these cells are xenogenic. Thus, their use may result in a severe immune response following implantation.

It is believed that a decellularized xenogenic tissue may be a viable option as a replacement tissue, as the antigenic cellular material will be removed while preserving the relatively nonimmunogenic extracellular matrix (ECM). Ideally, this will also preserve the biomechanical properties of the tissue. For instance, an acellular dermal matrix has seen successful use clinically as the FDA approved Alloderm product. Additionally, acellular xenogenic tissues have been created for many musculoskeletal applications, including replacements for the knee meniscus, temporomandibular joint disc, tendon, and ACL, as well as in other tissues including heart valves, bladder, artery, and small intestinal submucosa.

### SUMMARY

The present disclosure, according to certain embodiments, is generally in the field of improved methods for tissue engineering. More particularly, the present disclosure relates to methods for forming tissue engineered constructs without the use of scaffolds and that eliminate cells from the tissue engineered constructs intended for tissue replacement, which may be non-immunogenic. As used herein, a “construct” or “tissue engineered construct” refers to a three-dimensional mass having length, width, and thickness, and which comprises living mammalian tissue produced in vitro.

Prior studies have used SDS for tissue decellularization, but none have involved the use of scaffoldless tissue engineered constructs, or native cartilage tissue. The methods of the present disclosure provide the ability to decellularize custom engineered tissue to remove the immunogenicity of the tissue while maintaining the biochemical and biomechanical properties of the tissue. Engineered tissues custom designed to a defect, even up to a mold of the entire joint surface could be created from bovine or other animal cells, which have a nearly limitless supply, and could have properties tailored to the desired application prior to decellularization. For example, the engineered tissues custom designed to a defect may serve as a tissue replacement for joints, ear, nose, or other articular/non-articular cartilages.

The features and advantages of the present invention will be readily apparent to those skilled in the art upon a reading of the description of the embodiments that follows.

### DRAWINGS

The patent or application file contains at least one drawing executed in color. Copies of this patent or patent application publication with color drawing(s) will be provided by the Office upon request and payment of the necessary fee.

Some specific example embodiments of the disclosure may be understood by referring, in part, to the following description and the accompanying drawings.

FIG. 1A photomicrographs demonstrating construct cellularity, GAG content, and collagen content for various treatment groups. 10× original magnification. Treatment with 2% SDS for 1 h decreased cellularity while preserving GAG content, while treatment for 8 h eliminated all nuclei, but also eliminated all GAG.

FIG. 1B photomicrographs demonstrating construct cellularity, GAG content, and collagen content for treatment groups in phase II. 10× original magnification. Treatment with 2% SDS for 1, 2, and 4 h decreased cellularity while preserving GAG and collagen content, while treatment for 6 and 8 h eliminated all nuclei, but also eliminated GAG and reduced collagen.

FIG. 2A is a graph showing DNA content of constructs following decellularization treatment in phase I. Treatment with 2% SDS or the hypotonic/hypertonic solutions at either application time significantly decreased construct DNA content. Columns and error bars represent means and standard deviations. Groups denoted by different letters are significantly different (p<0.05).

FIG. 2B is a graph showing DNA content of constructs following decellularization treatment in phase II. Treatment with 2% SDS at all application times significantly reduced DNA content, while treatment for 8 h resulted in the greatest reduction in DNA content. Columns and error bars represent means and standard deviations. Groups denoted by different letters are significantly different (p<0.05).

FIG. 3 are graphs showing construct properties Construct biochemical properties following decellularization in phases I and II. (A) In phase I, all 8 h treatments resulted in nearly complete GAG removal, while both 1% and 2% SDS for 1 h maintained GAG content. (B) In phase I, treatment with SDS or TnBP maintained collagen content, while treatment with Triton X-100 or the hypotonic/hypertonic combination significantly reduced total collagen content. (C) In phase II, treatment for 1 or 2 h maintained GAG content, while treatment for 6 or 8 h resulted in near complete GAG removal. (D) In phase II, treatment for 1, 2, 4, or 6 h maintained collagen content, while treatment for 8 h resulted in a reduction in collagen content. Columns and error bars.

FIG. 4 are graphs showing construct biomechanical properties following decellularization in phases I and II. (A) In phase I, all 8 h treatments either significantly reduced aggregate modulus, or were untestable. Treatment for 1 h with 1% or 2% SDS, or 2% TnBP maintained aggregate modulus. (B) In phase I, treatment with 1% SDS for 1 h maintained Young's modulus, while treatment with 2% SDS for 1 h increased Young's modulus. (C) In phase II, 2% SDS treatment for 1 or 2 h maintained compressive properties, while treatment for 6 or 8 h resulted in constructs that were untestable in compression. (D) In phase II, treatment for 1, 2, or 4 h maintained Young's modulus, while 6 and 8 h treatments significantly reduced Young's modulus. (E) Similar trends were observed for ultimate tensile strength. Columns and error bars represent mean values and standard deviations. Groups denoted by different letters are significantly different (p<0.05).

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to certain embodiments, is generally in the field of improved methods for tissue engineering. More particularly, the present disclosure relates to methods for forming tissue engineered constructs without the use of scaffolds and that eliminate cells from the tissue engineered constructs intended for tissue replacement, which may be non-immunogenic. As used herein, a “construct” or “tissue engineered construct” refers to a three-dimensional mass having length, width, and thickness, and which comprises mammalian tissue produced in vitro.

The methods of this disclosure generally comprise the formation of a tissue engineered constructs without the use of scaffolds or other synthetic materials. Generally, cells are seeded on a shaped hydrogel mold and allowed to self-assemble to form a construct and the construct is decellularized. As used herein, “self-assemble” or “self-assembly” refers to a process in which specific local interactions and constraints between a set of components cause the components to autonomously assemble, without external assistance, into the final desired structure through exploration of alternative configurations.

The hydrogel used in conjunction with the methods of the present disclosure may comprise agarose, alignate, or combinations thereof. A “hydrogel” is a colloid in which the particles are in the external or dispersion phase and water is in the internal or dispersed phase. Suitable hydrogels are nontoxic to the cells, are non-adhesive, do not induce chondrocytic attachment, allow for the diffusion of nutrients, do not degrade significantly during culture, and are firm enough to be handled.

In particular embodiments, the hydrogel used in conjunction with the present disclosure is melted to form a molten hydrogel. The molten hydrogel is introduced into a culture vessel and may be shaped using a shaped press. The press may be shaped to accommodate the desired shape of the tissue engineered construct. For example, the press may be fashioned from a 3-dimensional scan of a total joint to result in molds the shape of this joint. Similarly, molds may be fashioned from 3-dimensional scanning of ear, nose, or other nonarticular cartilage to form molds the shape of these cartilages.

The cells used in conjunction with the methods of the present disclosure may be chondrocytes or chondrocytic type cells. The cells may be derived from a xenogenic source (e.g., from bovine or porcine cells). Another suitable source of cells is heterologous chondrocytes from cartilage tissue obtained from a donor or cell line. Examples of suitable cells include, but are not limited to, meniscal fibrochondrocytes, temporomandibular joint disc cells, mesenchymal stem cells, skin-derived cells, chondrocytes, fibrochondrocytes, and combinations thereof.

The cells may be cultured using any suitable means and conditions to produce a tissue-engineered construct. Choices in such means and conditions include, but are not limited to, the seeding concentration of the cell sample, the medium in which the cell sample is cultured, and the shape of the vessel in which the cell sample is cultured. The choice of such conditions may depend upon, among other things, the source of the cell sample and the desired size and shape of the tissue-engineered cartilage construct. One of ordinary skill in the art, with the benefit of this disclosure, will recognize suitable means and conditions for producing tissue-engineered cartilage constructs useful in the methods of the present invention. In certain embodiments, the culturing of the cells to produce a tissue-engineered construct may utilize a self-assembly process.

The cells seeded on hydrogel coated culture vessels or hydrogel negative molds are allowed to self-assemble. Self-assembly may result in the formation of non-attached constructs on the hydrogel surfaces. It is preferable to use hydrogel coated surfaces instead of tissue culture treated surfaces since articular chondrocytes seeded onto standard tissue culture treated plastic (TCP) readily attach, spread, and dedifferentiate.

In particular embodiments, the cells may be treated with staurosporine, a protein kinase C inhibitor and actin disrupting agent, during the self-assembly process to reduce synthesis of αSMA, a contractile protein. Reducing αSMA in the constructs via staurosporine treatment may reduce construct contraction and may also upregulate ECM synthesis.

In certain embodiments, the tissue-engineered construct may be treated by use of a biochemical reagent, a mechanical force, hydrostatic pressure, or any combination thereof.

The step of treating the tissue-engineered construct may be performed at any desired time, which may be during or after the tissue-engineered construct is produced. In certain embodiments, treating the tissue-engineered construct may comprise the use of a biochemical reagent, a mechanical force, hydrostatic pressure, or any combination thereof. Such treatments may, among other things, enhance the morphological, biochemical, and/or biomechanical properties of the treated tissue-engineered cartilage construct.

A variety of biochemical reagents may be used to treat the tissue-engineered constructs. Such biochemical reagents include any biochemical reagent suitable for enhancing the morphological, biochemical, and/or biomechanical properties of the treated tissue-engineered cartilage construct. Such suitable biochemical reagents may include, but are not limited to, gylcosaminoglycan (GAG) depleting agents, growth factors, and any combination thereof. Example of GAG depleting agents which may be suitable for use in the methods of the present invention are chondroitinase-ABC (C-ABC), aggrecanases, keratinases, and combinations thereof. An example of a growth factor which may be suitable for use in the methods of the present invention is transforming growth factor-β1 (TGF-β1). One of ordinary skill in the art, with the benefit of this disclosure, may recognize additional biochemical reagents that may be useful in the methods of the present invention. The biochemical reagents useful in the methods of the present invention may be used to treat the tissue-engineered cartilage constructs at any time during or after the production of the tissue-engineered cartilage construct. Such a choice of treatment time may depend upon, among other things, the desired degree of treatment and the specific biochemical reagent chosen. One of ordinary skill in the art, with the benefit of this disclosure, will be able to choose when to treat the tissue-engineered construct with the biochemical reagents useful in the methods of the present invention.

The mechanical force used in the methods of the present invention to treat the tissue-engineered construct may be applied in any amount and by any means suitable to enhance the morphological, biochemical, and/or biomechanical properties of the treated tissue-engineered cartilage construct. An example of a suitable mechanical force is direct compression. In certain embodiments, the choice of an appropriate mechanical force may comprise the selection of an appropriate strain and frequency. Such a choice of strain and frequency may depend upon, among other things, the size and shape of the tissue-engineered cartilage construct. One of ordinary skill in the art, with the benefit of this disclosure, will recognize suitable strains and frequencies that may be useful in the methods of the present invention.

In certain embodiments, the use of mechanical force may comprise the use of a strain of 7 to about 17% and a frequency of 0 to about 1 Hz. In certain embodiments, such mechanical force may be applied from 1 to 4 days after production of the tissue-engineered construct in 60 second cycles (i.e. 60 seconds of mechanical force, followed by 60 seconds of no mechanical force) for about 1 hour total mechanical force application per day. By way of explanation, and not of limitation, such a mechanical force treatment may, among other things, increase one or more of the wet weight (ww), thickness, and ratio of GAG concentration to wet weight (GAG/ww) of the tissue-engineered cartilage construct.

In certain embodiments, the mechanical force treatment may be applied with a varying (i.e. non-repetitive) manner, such as varying periods in which no mechanical force is applied. In certain embodiments, the mechanical force may be applied on non-consecutive days. In certain embodiments, the mechanical force may be applied at differing strains ranging from about 0.1% to about 99%. In certain embodiments, mechanical forces of various magnitudes may be applied during the same treatment. Such variations in the mechanical force treatment, among other things, may aid in the enhancement of the morphological, biochemical, and/or biomechanical properties of the treated tissue-engineered cartilage construct.

The hydrostatic pressure (HP) used in the methods of the present invention to treat the tissue-engineered construct may be applied in any amount and by any means suitable to enhance the morphological, biochemical, and/or biomechanical properties of the treated tissue-engineered cartilage construct. In certain embodiments, the HP used in the methods of the present invention may be static HP. In certain embodiments, the choice of an appropriate HP may comprise the choice of an appropriate magnitude and duration of HP treatment. One of ordinary skill in the art, with the benefit of this disclosure, will recognize suitable magnitudes and durations of HP treatment that may be useful in the methods of the present invention.

In certain embodiments, the use of hydrostatic pressure to treat the tissue-engineered construct may comprise the use of 10 MPa static HP for 1 hour/day for a 5-day period before or after the production of the tissue-engineered construct. In certain embodiments, such a hydrostatic pressure treatment may increase one or more of the aggregate modulus, the Young's modulus, the ratio of GAGs to wet weight (GAG/ww), and the ratio of collagen to wet weight (collagen/ww).

In certain embodiments, hydrostatic pressure may be applied repeatedly on non-consecutive days. In certain embodiments, hydrostatic pressure may be applied multiple times per day, optionally with varying periods in which no hydrostatic pressure is applied. In certain embodiments, the magnitude of the hydrostatic pressure may range from about 0.01 to about 20 MPa. In certain embodiments, varying magnitudes of hydrostatic pressure may be utilized in the same treatment. In certain embodiments, non-static HP may be employed, optionally at varying frequencies. In certain embodiments, such non-static HP treatments may have a sinusoidal pattern of magnitude.

In certain embodiments, the cells used in conjunction with the methods of the present disclosure may be seeded on a hydrogel coated culture vessel and allowed to self-assemble before being transferred to a shaped hydrogel negative mold. Alternatively, rather than seeding the cells on a hydrogel coated culture vessel, in certain embodiments, the cells may be seeded directly onto a shaped hydrogel negative mold. The shaped hydrogel negative mold may comprise agarose. Other non-adhesive hydrogels, e.g. alignate, may be used in conjunction with the methods of the present disclosure. In other embodiments, the hydrogel mold may be a two piece structure comprising, a shaped hydrogel negative mold and a shaped hydrogel positive mold. The shaped hydrogel negative and positive molds may comprise the same non-adhesive hydrogel or may be a comprised of different non-adhesive hydrogels. In certain embodiments, the cells may be seeded on a hydrogel coated culture vessel and allowed to self-assemble into a first construct. The first construct may be transferred to a shaped hydrogel negative mold. A shaped hydrogel positive mold may be applied to the negative mold to form a mold-construct assembly. The mold-construct assembly may then further be cultured to form a second construct. As used herein, the term “mold-construct assembly” refers to a system comprising a construct or cells within a shaped positive and a shaped negative hydrogel mold.

In certain embodiments, the molds may be shaped from a 3-D scanning of a total joint to result in a mold fashioned in the shape of said joint. In other embodiments, the molds may be shaped from a 3-D scanning of the ear, nose, or other non-articular cartilage to form molds in the shapes of these cartilages. In certain embodiments, the mold may be shaped to be the same size as the final cartilaginous product. In other embodiments, the molds may be shaped to be smaller than the final cartilaginous product. In certain embodiments, the molds may be fashioned to a portion of a joint or cartilage so that it serves as a replacement for only a portion of said joint or cartilage.

According to the methods of the present disclosure, once the tissue-engineered construct is formed it is decellularized to substantially remove any cells that may be present while maintaining biomechanical properties. Accordingly, the methods of the present invention also include decellularizing the tissue-engineered construct. The decelluarization generally comprises contacting the tissue-engineered construct with a compound chosen from one or more of a detergent, an organophosphorus compound, and a surfactant at a concentration and time sufficient to substantially remove any cells that may be present. Examples of suitable decellularizing compounds include, but are not limited to, detergents such as sodium dodecyl sulfate, organophosphorus compounds such as tributyl phosphate, and surfactants such as polyethylene glycol p-(1,1,3,3-tetramethylbutyl)-phenyl ether.

In certain embodiments, decellularizing the tissue-engineered construct may further include contacting the tissue-engineered construct with a nuclease, a proteinase, an antibiotic, and an antifungal. In other embodiments, the decellularization may further include introducing the tissue-engineered construct into a solution comprising phosphate buffered saline or culture media at 37° C. with or without agitation; and washing the tissue-engineered construct in the solution to substantially remove the detergent, the organophosphorus compound, or the surfactant.

In some embodiments, complete decellularization is not required. Instead, decelluarization need only be sufficient to eliminate an immune response.

To facilitate a better understanding of the present invention, the following examples of specific embodiments are given. In no way should the following examples be read to limit or define the entire scope of the invention.

### EXAMPLES

### Materials and Methods

Chondrocyte Isolation and Seeding

Cartilage was harvested from the distal femur of wk-old male calves (Research 87, Boston, Mass.) shortly after slaughter, and chondrocytes were isolated following digestion with collagenase type 2 (Worthington, Lakewood, N.J.). To normalize variability among animals, each leg came from a different animal, and cells from all legs were combined together to create a mixture of chondrocytes; a mixture of cells from five legs was used in the study. Cell number was determined on a hemocytometer, and a trypan blue exclusion test indicated that viability remained>90%. Chondrocytes were frozen in culture medium supplemented with 20% FBS (Biowhittaker, Walkersville, Md.) and 10% DMSO at −80° C. for 1 day prior to use. After thawing, viability was greater than 90%. A stainless steel mold consisting of 5 mm dia. ×10 mm long cylindrical prongs was placed into a row of a 48-well plate. To construct each agarose well, sterile, molten 2% agarose was added to wells fitted with the die. The agarose solidified at room temperature for 60 min, after which the mold was removed from the agarose. Two changes of culture medium were used to completely saturate the agarose well by the time of cell seeding. The medium was DMEM with 4.5 g/L-glucose and L-glutamine (Biowhittaker), 100 nM dexamethasone (Sigma, St. Louis, Mo.), 1% Fungizone/Penicillin/Streptomycin (Biowhittaker), 1% ITS+(BD Scientific, Franklin Lakes, N.J.), 50 μg/mL ascorbate-2-phosphate, 40 μg/mL L-proline, and 100 μg/mL sodium pyruvate (Fisher Scientific, Pittsburgh, Pa.). To seed each construct, 5.5×10⁶ cells were added in 100 μl of culture medium. Constructs formed within 24 h in the agarose wells and were cultured in the same well until t=10 days, after which they were unconfined for the remainder of the study, as described previously; t=0 was defined as 24 h after seeding. Throughout the studies, constructs were cultured in an incubator at 37° C. and 10% CO₂.

Decellularization Treatments Phase I

At t=4 wks, self-assembled constructs (n=6/group) were removed from culture and exposed to one of five decellularization treatments, for either 1 h or 8 h. The decellularization treatments included:

1) 1% SDS

2) 2% SDS

3) 2% Tributyl Phosphate (TnBP)

4) Triton X-100 (polyethylene glycol p-(1,1,3,3-tetramethylbutyl)-phenyl ether)

5) Hypotonic/Hypertonic Solution (half-time of each)
a. Hypotonic: 10 mM Tris HCl, 5 mM EDTA, 1 μM PMSF
b. Hypertonic: 50 mM Tris HCl, 1 M NaCl, 10 mM EDTA, 1 μM PMSF

All treatments included 0.5 mg/ml DNase Type I, 50 μg/ml RNase, 0.02% EDTA, and 1% P/S/F, in PBS. Both 1 h control and 8 h control groups were exposed to this same solution without detergent treatments. These treatments were applied at 37° C. with agitation. Following the 1 h or 8 h treatment, the constructs were washed for 3 h in PBS at 37° C. with agitation. Additionally, an untreated control was assessed immediately following removal from culture, without the treatment or wash steps.

Decellularization Treatments Phase II

At t=4 wks, self-assembled constructs (n=6/group) were removed from culture and exposed to 2% SDS for 1, 2, 4, 6, or 8 h. As in phase I, all treatments included 0.5 mg/mL DNase Type I, 50 μg/mL RNase, 0.02% EDTA, and 1% P/S/F, in PBS. These treatments were applied at 37° C. with agitation. Following the SDS treatment, the constructs were washed for 2 h in PBS at 37° C. with agitation. Additionally, an untreated control was assessed immediately following construct removal from culture, without the treatment or wash steps.

Histology and Immunohistochemistry

After freezing, samples were sectioned at 14 μm. To determine construct cellularity, a hematoxylin & eosin (H&E) stain was used. A Safranin-O/fast green stain was used to examine GAG distribution, and picrosirius-red was employed for collagen content.

Immunohistochemistry was utilized to test for the presence of collagen types I and II on a Biogenex (San Ramon, Calif.) i6000 autostainer. Following fixation in chilled acetone, the slides were washed with IHC buffer (Biogenex), quenched of peroxidase activity with hydrogen peroxide/methanol, and blocked with horse serum (Vectastain ABC kit, Vector Laboratories, Burlingame, Calif.). The slides were then incubated with either mouse anti-collagen type I (Accurate Chemicals, Westbury, N.Y.) or rabbit anti-collagen type II (Cedarlane Labs, Burlington, N.C.) antibodies. Secondary antibody (anti-mouse or anti-rabbit IgG, Vectastain ABC kit) was applied, and color was developed using the Vectastain ABC reagent and DAB (Vectastain kit).

Quantitative Biochemistry

Samples were frozen overnight and lyophilized for 48 h, followed by re-suspension in 0.8 mL of 0.05 M acetic acid with 0.5 M NaCl and 0.1 mL of a 10 mg/mL pepsin solution (Sigma) at 4° C. for 72 h. Next, 0.1 mL of 10×TBS was added along with 0.1 mL pancreatic elastase and mixed at 4° C. overnight. A Picogreen® Cell Proliferation Assay Kit (Molecular Probes, Eugene, Oreg.) was used to assess total DNA content. GAG content was quantified using the Blyscan Glycosaminoglycan Assay kit (Biocolor), based on 1,9-dimethylmethylene blue binding. After hydrolysis with 2 N NaOH for 20 min at 110° C., total collagen content was determined using a chloramine-T hydroxyproline assay.

Indentation Testing

Samples were assessed with an automated indentation apparatus, as described previously. A 0.7 g (0.007 N) mass was applied with a 1 mm flat-ended, porous indenter tip, and specimens crept until equilibrium, as described elsewhere. Preliminary estimations of the aggregate modulus of the samples were obtained using the analytical solution for the axisymmetric Boussinesq problem with Papkovich potential functions. The sample biomechanical properties, including aggregate modulus, Poisson's ratio, and permeability were then calculated using the linear biphasic theory.

Tensile Testing

A uniaxial materials testing system (Instron Model 5565, Canton, Mass.) was employed to determine tensile properties with a 50 N load cell, as described previously. Briefly, samples were cut into a dog-bone shape with a 1-mm-long gauge length. Samples were glued to paper tabs with cyanoacrylate glue outside of the gauge length. The 1-mm-long sections were pulled at a 1% constant strain rate. All samples broke within the gauge length. The gauge length, thickness, and initial cross-sectional area were measured using digital calipers. For each construct, a stress-strain curve was created from the load-displacement curve and Young's modulus was calculated from each stress-strain curve using the initial cross-sectional area.

Statistical Analysis

All samples were assessed biochemically and biomechanically (n=6). First, the three control groups were compared using a single factor ANOVA. As no difference was noted, only the culture control was used in the final analysis. To compare treatment groups in both phases, a single factor ANOVA was used, and a Tukey HSD post hoc test was used when warranted. Significance was defined as p<0.05.

Results

Gross Appearance and Histology

In all groups, the construct diameter was approximately 6 mm at 4 wks. In phase I, treatment for 8 h with either 1% SDS or the hypotonic/hypertonic solution resulted in a significant decrease in construct thickness (Table 1). Additionally, treatment for 8 h with 1% SDS, 2% SDS, 2% Triton X-100, or the hypotonic/hypertonic solution resulted in a significant decrease in construct wet weight (Table 1). In phase II, treatment with 2% SDS for 6 h or 8 h resulted in a significant decrease in construct thickness and wet weight (Table 2).

TABLE 1
Phase I. Construct wet weight and thickness values.

| Treatment | Construct Wet | Thickness |
|---|---|---|
| Group | Weight (mg) | (mm) |
| Control | 14.8 ± 1.1 | 0.49 ± 0.03 |
| 1% SDS, 1 h | 14.3 ± 1.0 | 0.50 ± 0.02 |
| 1% SDS, 8 h | 8.8 ± 1.2^{a} | 0.38 ± 0.04^{a} |
| 2% SDS, 1 h | 12.3 ± 1.1 | 0.43 ± 0.05 |
| 2% SDS, 8 h | 9.3 ± 2.6^{a} | 0.47 ± 0.08 |
| 2% TnBP, 1 h | 15.2 ± 1.1 | 0.53 ± 0.06 |
| 2% TnBP, 8 h | 12.2 ± 1.2 | 0.49 ± 0.04 |
| 2% Triton X-100, 1 h | 13.7 ± 1.2 | 0.47 ± 0.05 |
| 2% Triton X-100, 8 h | 11.2 ± 1.7^{a} | 0.47 ± 0.08 |
| Hypo/Hyper 1 h | 15.0 ± 3.0 | 0.40 ± 0.09 |
| Hypo/Hyper 8 h | 7.0 ± 1.3^{a} | 0.35 ± 0.04^{a} |

^{a}Significantly lower than control (p < 0.05)

TABLE 2
Phase II. Construct wet weight and thickness values.

| Treatment | Construct Wet | Thickness |
|---|---|---|
| Group | Weight (mg) | (mm) |
| Control | 19.9 ± 3.3 | 0.73 ± 0.14 |
| 1 h | 16.0 ± 4.1 | 0.73 ± 0.16 |
| 2 h | 15.8 ± 3.6 | 0.66 ± 0.10 |
| 4 h | 14.8 ± 2.5 | 0.56 ± 0.09 |
| 6 h | 9.3 ± 1.9^{a} | 0.53 ± 0.07^{a} |
| 8 h | 10.7 ± 1.8^{a} | 0.53 ± 0.08^{a} |

^{a}Significantly lower than control (p < 0.05)

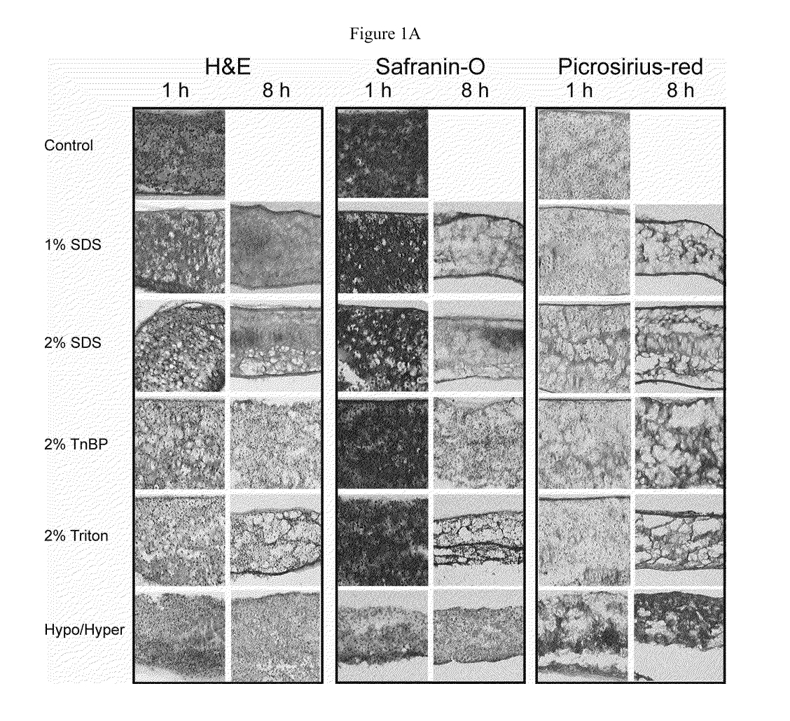

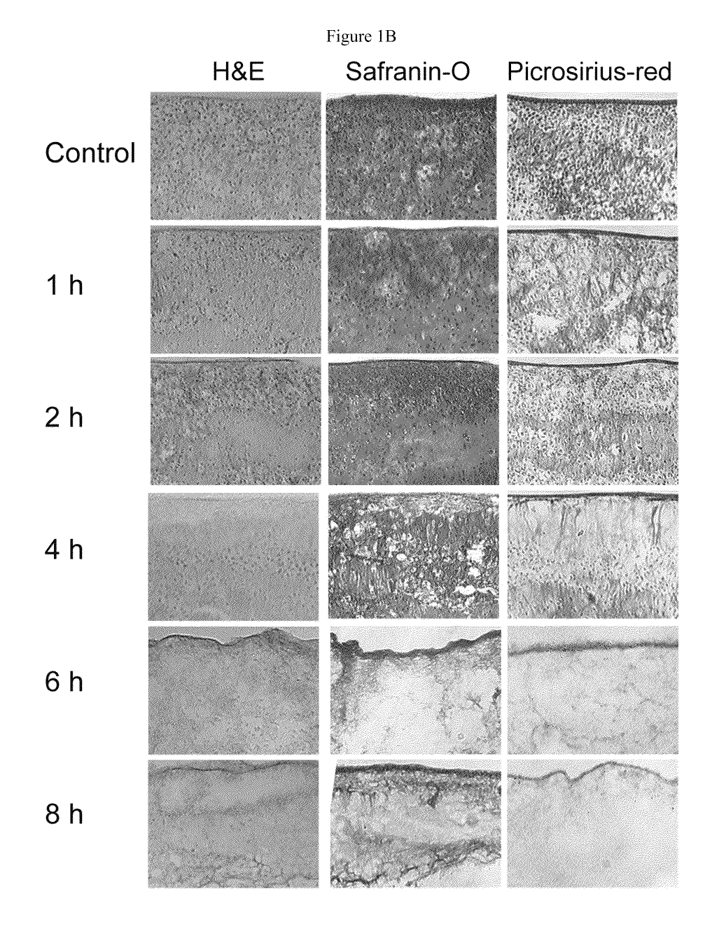

FIG. 1A displays the histological results of Phase 1. Extensive staining for cell nuclei was observed in the H&E staining of the control group. Treatment with 1% SDS treatment for 1 h reduced the number of cell nuclei, while treatment for 8 h eliminated all nuclei from the construct. The 2% SDS treatment had similar results. However, treatment with 2% TnBP or 2% Triton X-100, for either timepoint, had no effect on the number of nuclei. Both hypotonic/hypertonic treatments resulted in a slight reduction in number of cell nuclei. All decellularization treatments for 8 h resulted in a significant reduction or complete elimination of staining for GAGs. Additionally, 1 h treatment with the hypotonic/hypertonic solution reduced the GAG content. However, there were no apparent differences in GAG staining among the 1 h treatments with 1% SDS, 2% SDS, 2% TnBP, 2% Triton X-100, and the control. Finally, all constructs demonstrated extensive staining for collagen.

FIG. 1B displays the histological results of phase II. Extensive staining for cell nuclei was observed in the H&E staining of the control group. Increasing decellularization was observed with 2% SDS treatment from 1 to 4 h, while 6 or 8 h application times were required for complete histological decellularization. Treatment for 1 and 2 h resulted in maintenance of GAG and collagen staining, while the 4 h treatment resulted in decreased staining. However, treatment for 6 and 8 h resulted in no GAG staining and poor collagen staining.

Quantitative Biochemistry

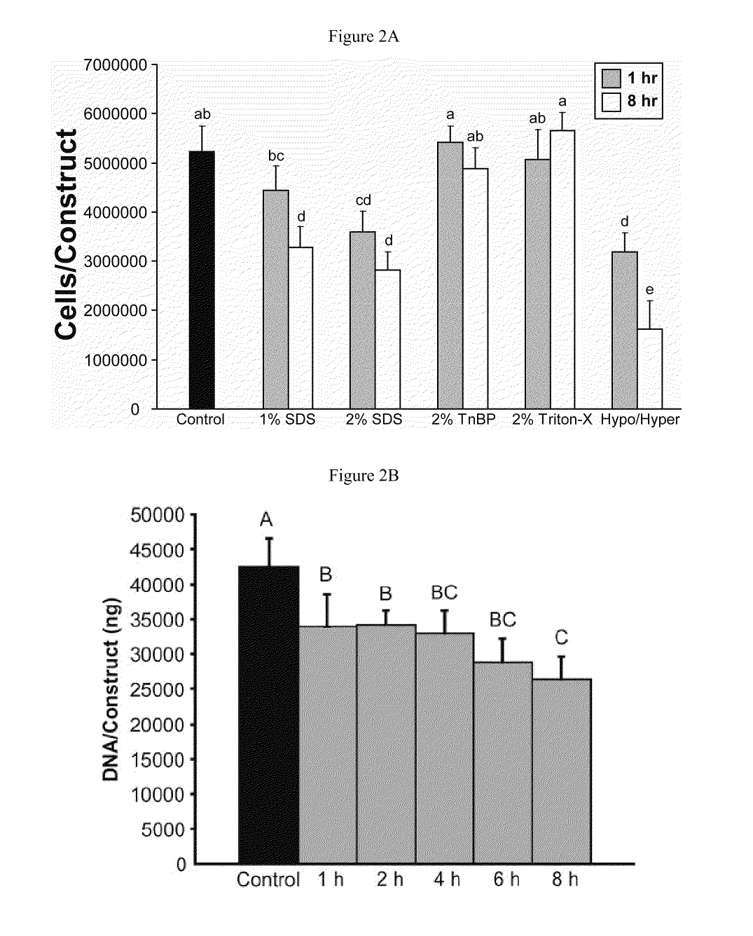

In phase I, several decellularization treatments resulted in a significant reduction in construct DNA (FIG. 2A). Treatment for 1 h with 2% SDS or the hypotonic/hypertonic solution, as well as 8 h treatment with 1 or 2% SDS or the hypotonic/hypertonic solution all resulted in a significant reduction of the DNA in the constructs. However, treatment with 2% TnBP or 2% Triton X-100 for either amount of time had no effect on construct DNA. In phase II, all application times resulted in a significant decrease in DNA content, although treatment for 8 h resulted in the greatest decrease (FIG. 2B).

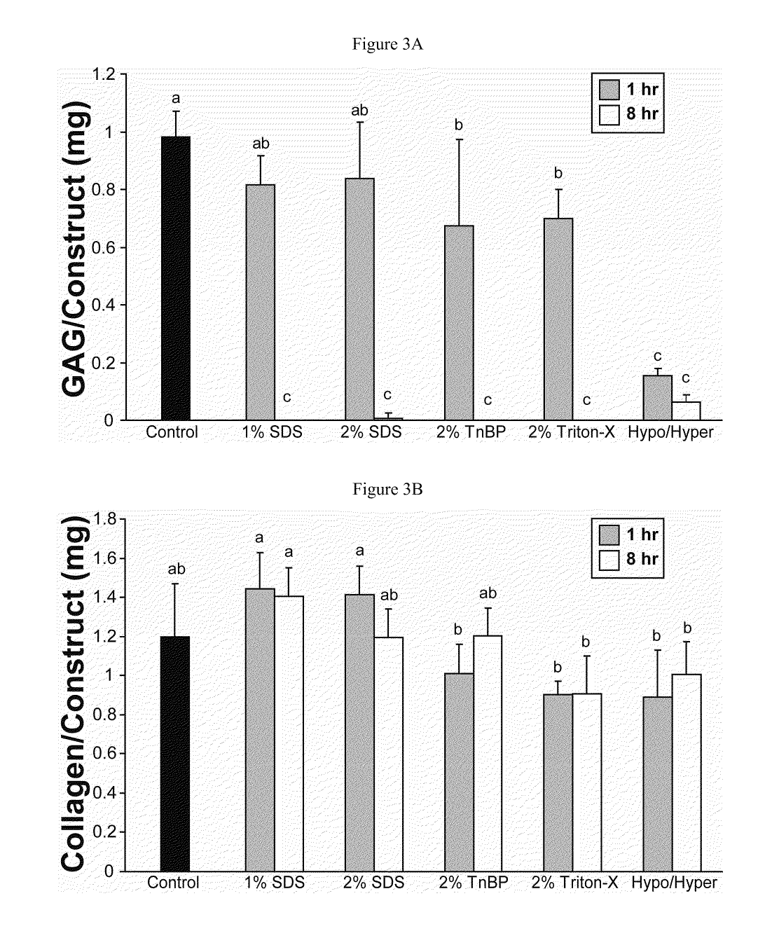

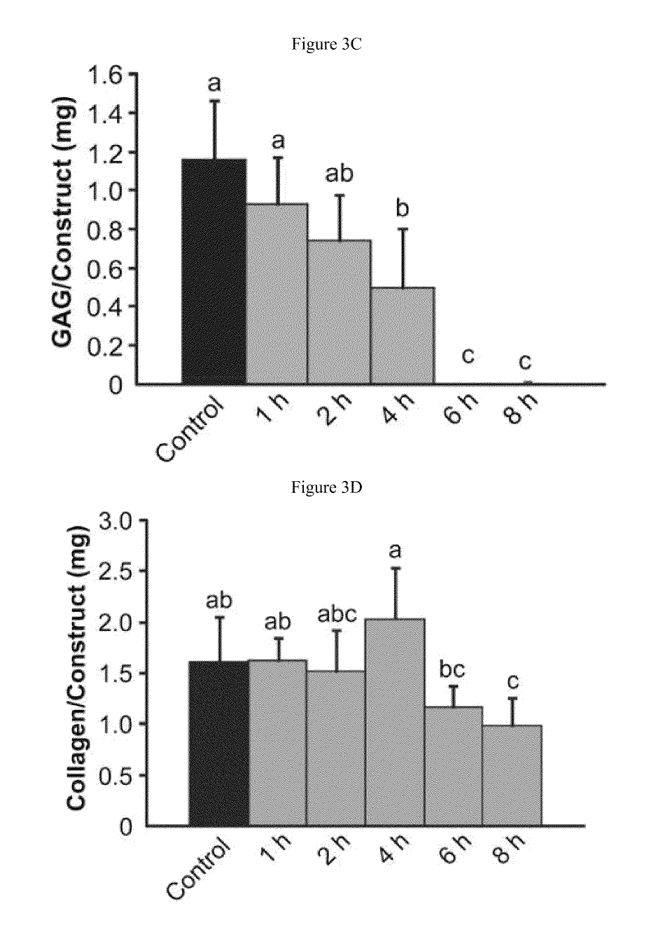

For phase I, the effects of the decellularization agents on construct GAG content are found in FIG. 3A. Treatment with 1% or 2% SDS for 1 h had no effect on GAG content, while all other treatments significantly reduced the GAG content of the constructs. Additionally, all 8 h treatments resulted in complete or nearly complete removal of GAG from the constructs. Finally, there were no significant changes in total collagen content following treatment with the decellularization agents (FIG. 3B). For phase II, the effects of the decellularization agents on construct GAG content are found in FIG. 3C. Treatment with 2% SDS for 1 or 2 h maintained GAG content, while 4 h treatment resulted in a significant decrease in GAG content. However, treatment for 6 or 8 h resulted in complete elimination of GAG. Treatment for 1, 2, 4, or 6 h did not significantly alter the collagen content, while treatment for 8 h resulted in a slight decrease in collagen content, as shown in FIG. 3D.

Biomechanical Evaluation

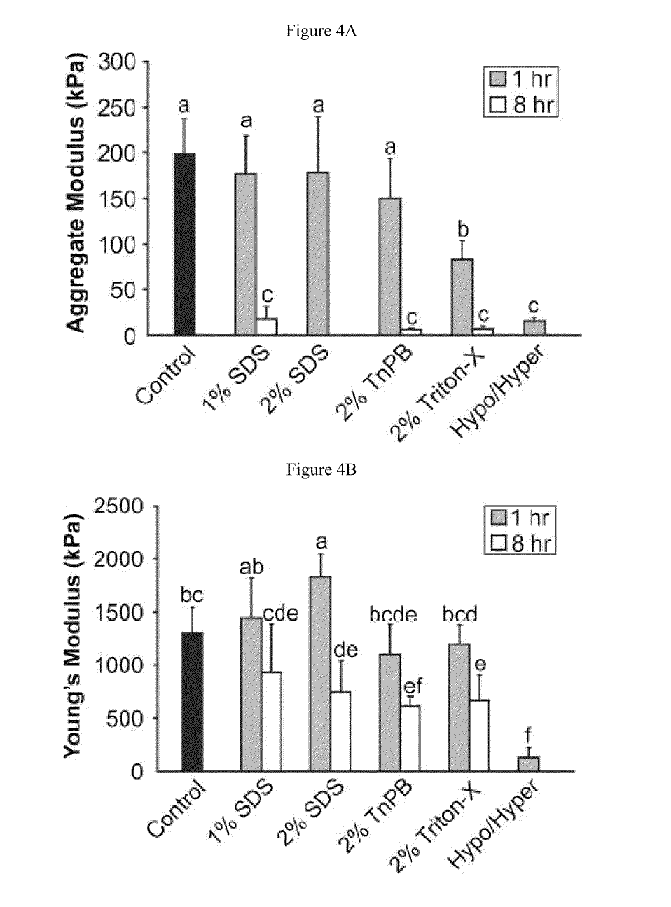

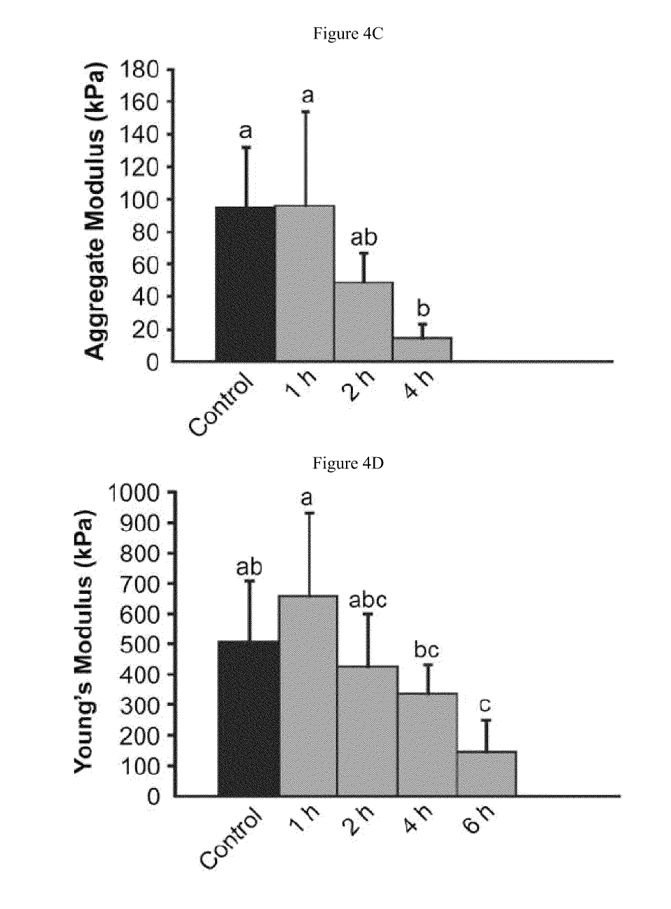

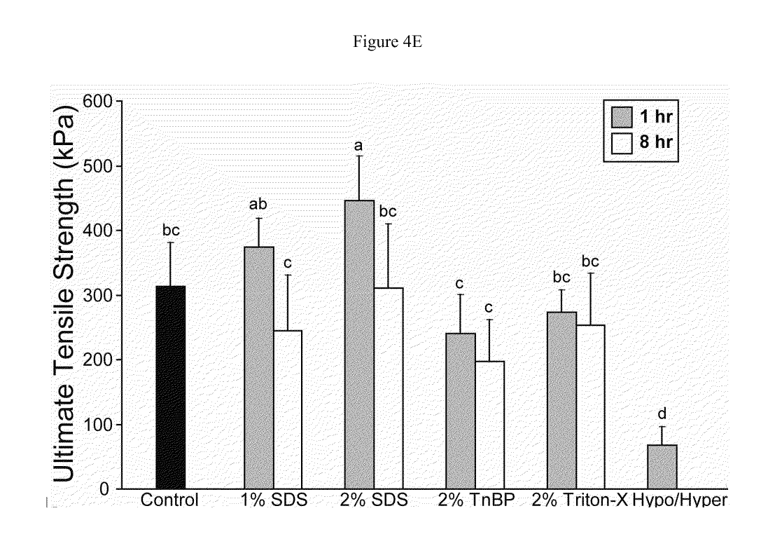

For phase I, the effects of the various decellularization treatments on construct aggregate modulus are displayed in FIG. 4A. Treatment for 1 h with 1% or 2% SDS as well as with 2% TnBP maintained the compressive stiffness. However, treatment for 8 h with 1% SDS, 2% TnBP, and 2% Triton X-100 significantly reduced the aggregate modulus. The groups treated for 8 h with either 2% SDS or the hypotonic/hypertonic solutions were too weak to be mechanically tested with creep indentation. Additionally, the effects of the various decellularization treatments on Poisson's ratio and permeability are displayed in Table 3. A significant decrease in Poisson's ratio was noted for the groups treated for 8 h with 1% SDS, 2% TnBP, and 2% Triton X-100. Finally, only treatment for 8 h with 1% SDS resulted in a significantly decreased permeability. FIG. 4B indicates the tensile properties of the constructs treated with the various agents in phase I. Treatment for 1 h with 1% SDS, 2% TnBP, or 2% Triton X-100 maintained Young's modulus. A 1 h treatment with 2% SDS actually increased Young's modulus. However, 8 h treatments with 2% SDS, 2% TnBP, and 2% Triton X-100 significantly decreased Young's modulus.

TABLE 3
Phase I values of Poisson ratio and permeability
following decellularization.

| Treatment Group | Poisson Ratio | Permeability |
|---|---|---|
| Control | 0.30 ± 0.07 | 14.3 ± 3.9 |
| 1% SDS, 1 h | 0.26 ± 0.04 | 15.6 ± 8.0 |
| 1% SDS, 8 h | 0.07 ± 0.09^{a} | 2.0 ± 1.6^{a} |
| 2% SDS, 1 h | 0.26 ± 0.10 | 12.6 ± 6.3 |
| 2% SDS, 8 h | Not testable | Not testable |
| 2% TnBP, 1 h | 0.24 ± 0.13 | 5.5 ± 3.1 |
| 2% TnBP, 8 h | 0.04 ± 0.03^{a} | 7.3 ± 7.5 |
| 2% Triton X-100, 1 h | 0.16 ± 0.11 | 4.3 ± 2.6 |
| 2% Triton X-100, 8 h | 0.04 ± 0.04^{a} | 5.1 ± 4.7 |
| Hypo/Hyper 1 h | 0.14 ± 0.14 | 14.9 ± 6.6 |
| Hypo/Hyper 8 h | Not testable | Not testable |

^{a}Significantly lower than control (p < 0.05)

For phase II, the effects of the various application times on construct aggregate modulus are displayed in FIG. 4C. There was no significant difference in aggregate modulus with treatment for 1 and 2 h, while the 4 h treatment significantly reduced the stiffness. Additionally, the 6 and 8 h treatment resulted in constructs that were untestable in compression. As shown in Table 4, the 1, 2, and 4 h treatments did not result in significant changes in permeability and Poisson's ratio. FIG. 4D displays the tensile properties of the constructs treated in phase II. Treatment with 2% SDS for 1 h resulted in a slight increase in tensile properties, although this was not significant. Treatment for 2 and 4 h maintained Young's modulus while treatment for 6 h resulted in a reduced Young's modulus. Constructs treated for 8 h were untestable in tension.

TABLE 4
Phase II values of Poisson ratio and permeability
following decellularization.

| Treatment | | |
|---|---|---|
| Group | Poisson Ratio | Permeability |
| Control | 0.13 ± 0.07 | 32.0 ± 18.2 |
| 1 h | 0.09 ± 0.08 | 27.0 ± 15.2 |
| 2 h | 0.08 ± 0.08 | 15.5 ± 4.4 |
| 4 h | 0.09 ± 0.09 | 66.3 ± 77.3 |
| 6 h | Not testable | Not testable |
| 8 h | Not testable | Not testable |

^{a}Significantly lower than control (p < 0.05)

The objective of this study was to assess the effectiveness of multiple different decellularization protocols on self-assembled articular cartilage constructs, and to determine an appropriate application time for the treatment, among other things. A two-phased approach was used. In phase I, a two-factor approach was employed, in which five different treatments were examined at two application times each. In phase II, the effects of multiple treatment times were examined.

The results of this study indicated that SDS, at concentrations of either 1% or 2%, is an effective treatment for tissue decellularization, thus confirming our hypothesis that cells could be eliminated from engineered constructs while maintaining the biomechanical properties. An ionic detergent, SDS typically is able to solubilize the nuclear and cytoplasmic cell membranes. Although all SDS treatments led to cell removal, treatment with 2% SDS appeared the most promising, although application time also had significant effects. For instance, treatment with 2% SDS for 1 h resulted in a 33% decrease in cellularity, while maintaining both GAG and collagen content, as well as maintaining compressive stiffness. This treatment even resulted in an increase in tensile stiffness; a similar increase in tensile properties was observed in a study of ACL decellularization. On the other hand, treatment with 2% SDS for 8 h led to complete histological decellularization, as well as a 46% decrease in DNA content. However, this treatment also resulted in loss of all GAG and compressive stiffness, as well as a decrease in tensile stiffness. Treatment with 2% SDS for 8 h also resulted in a significant decrease in construct wet weight, presumably as a result of the GAG loss, which would also decrease the tissue hydration. As 2% SDS for 8 h resulted in the greatest decrease in DNA content, and treatment for 1 h maintained or increased biomechanical and biochemical properties, 2% SDS was selected for use in phase II.

The assessed histological, biochemical and biomechanical properties of the untreated tissue engineered constructs are in the range of the starting immature bovine cartilage, although the tensile properties are only about 10-15% of native tissue. For instance, the aggregate modulus of immature bovine cartilage is 252±31, Young's modulus is 7.2±4.6 MPa, the GAG/WW is 0.04±0.03 mg/mg, and the collagen/WW is 0.13±0.01 mg/mg. Additionally, the constructs treated for 1 h with 1% SDS, 2% SDS, and 2% TnBP had an aggregate modulus, GAG/WW, and collagen/WW in the range of native tissue. However, the tensile properties of the tissue are lacking compared to those of native tissue. Therefore, 2% SDS treatment for 1 h, with a significant increase in Young's modulus, results in a value closer to that of native tissue. Additionally, it is important to note that ‘control’ constructs from our prior tissue engineering studies were used as the starting point in this study, due to ease of use; however, with the use of growth factor application and mechanical stimulation such as hydrostatic pressure, we have achieved an aggregate modulus, GAG/WW, and collagen/WW matching those of native tissue, and Young's modulus approaching 50% of that of native tissue. It is believed that the aggregate modulus and Young's modulus likely will be the most important properties to match to native tissue in future tissue engineering approaches. Similar biomechanical properties between the implanted construct and the surrounding native tissue will prevent added stress at the interface site. The Poisson ratio, a measure of the tissue's apparent compressibility, and the permeability, a measure of the resistance to fluid flow, should also approach native tissue values in order to achieve similar deformations and fluid movement under joint loading.

Treatment with 2% SDS for 1 h resulted in tissue decellularization while maintaining construct functional properties. Although SDS at all application times led to decellularization, 6 or 8 h were required for complete histological decellularization. However, these time points resulted in complete removal of GAG as well as an extremely poor aggregate modulus. However, the reduction in collagen content and tensile properties was less pronounced. On the other hand, as in phase I, treatment for 1 h resulted in a significant reduction in DNA content, while maintaining all biochemical and biomechanical properties, and even increasing Young's modulus. The observed increase in Young's modulus with a 1 h application of SDS suggests an effect of the detergent on collagen fibers within the engineered construct. SDS is known to have a propensity to disrupt non-covalent bonds in proteins and confer negative charges on proteins that have been denatured. The application of SDS for 1 h followed by a wash step may have had a transient effect on collagen architecture, wherein collagen fibers unfold as described previously, and then return to their native conformations, reforming non-covalent bonds and strengthening interactions in the process. The putative mechanism may have led to the observed increased Young's modulus at 1 h. With greater time in SDS, the effect is not observed, suggesting that any recovery undergone by collagen is counterbalanced by the detergent's aggregate effect on the rest of the tissue architecture.

It must be noted that although treatment with 2% SDS for 6 or 8 h resulted in complete histological decellularization, it did not result in complete elimination of DNA, which would be defined as ‘complete decellularization.’ It appeared that SDS treatment was effective at achieving complete lysis of cell membranes and nuclear membranes, as H&E staining did not reveal any indication of the presence of cell nuclei, while the DNase treatment was not completely effective in degrading the DNA following membrane lysis. It is possible that a higher DNase concentration is required to achieve more complete elimination of DNA. Additionally, as nucleases were only added during detergent treatment, it is possible that adding a nuclease during the wash step would enable the nucleases to more effectively destroy the remaining DNA.

However, the exact level of tissue decellularization requisite to eliminate an immune response, as well as the proper assessment of decellularization, is currently unclear. For instance, a recent study by Gilbert et al. demonstrated that several commercially available ECM scaffold materials contained measurable amounts of DNA; some even demonstrated histological staining for nuclear material. Most of these products have been used successfully clinically, so it is possible that having some remnant DNA and nuclear material in engineered cartilage constructs may result in a limited host response, though of course this needs to be demonstrated in vivo studies. Additionally, as it is believed that the joint space is relatively immune privileged, as reviewed previously it is possible that complete decellularization of the tissue is not required. Furthermore, it is unclear if decellularization should be assessed histologically merely as elimination of cell nuclei, or if a more complete assessment involves quantifying the tissue's DNA content, as prior studies have utilized differing approaches. For example, Lumpkins et al. [8] found that 1% SDS treatment for 24 h resulted in complete removal of cell nuclei, although they did not assess the DNA content of the tissue. On the other hand, Dahl et al. examined the effects of a hypotonic/hypertonic treatment and found that there was complete removal of cell nuclei, but no decrease in DNA content.

A drawback of using a decellularized xenograft is that it lacks chondrocytes, which are essential for the homeostasis of cartilage tissue. Eliminating the cells from the tissue leaves the ECM, which is responsible for the biomechanical properties of the tissue. Additionally, it has previously been demonstrated that decellularized bovine cartilage remained intact when implanted in a sheep for up to 6 months, and that there was cell infiltration, possibly from surrounding bone marrow MSCs. Therefore, it is possible that bone marrow infiltration of the decellularized constructs after implantation will allow for long term viability.

Although it was less effective than the 2% concentration, 1% SDS displayed similar effects. For example, treatment for 1 h resulted in a 15% decrease in DNA content, while maintaining GAG and collagen content as well as maintaining biomechanical properties. Additionally, treatment for 8 h resulted in a 37% decrease in DNA content, loss of all GAG and aggregate modulus, as well as a decrease in Young's modulus.

On the other hand, treatment with Triton X-100 and TnBP did not appear promising, as they had a minimal effect on tissue decellularization, and resulted in a slight decrease in GAG content. Several prior studies have indicated the ineffectiveness of Triton X-100, although it was used in this study as it is believed to have minimal effects on protein-protein interactions. For example, Dahl et al. examined the effects of 1% Triton X-100 on porcine carotid arteries, and found that this treatment resulted in similar cellularity to control and no decrease in DNA content. In another study on tendon decellularization, Cartmell and Dunn examined the effect of 1% Triton X-100 for 24 h, and found that cell density remained similar to control. Contrary to our results, this study demonstrated complete decellularization with 1% TnBP, although a 48 h treatment was required. Therefore, it is possible that TnBP treatment may result in decellularization of self-assembled constructs at longer application times, although the GAG loss after as little as 8 h prevents the use of longer application times.

Finally, although a hypotonic/hypertonic treatment has been an effective decellularization agent in this study as well as prior studies, it did not appear to be a viable treatment for self-assembled cartilage constructs, as it had severely detrimental effects on construct functional properties. For instance, treatment for as little as 1 h resulted in nearly complete loss of compressive and tensile stiffness, while constructs treated for 8 h were untestable mechanically. Additionally, treatment at both application times resulted in nearly complete elimination of GAG content.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

The following references are all incorporated by reference to the extent they provide information available to one of ordinary skill in the art regarding the implementation of the technical teachings of the invention.
[1] Buckwalter J A. Articular cartilage: injuries and potential for healing. J Orthop Sports Phys Ther 1998; 28(4):192-202.
[2] Hu J C, Athanasiou K A. A self-assembling process in articular cartilage tissue engineering. Tissue Eng 2006; 12(4):969-79.
[3] Elder B D, Athanasiou K A. Synergistic and additive effects of hydrostatic pressure and growth factors on tissue formation. PLoS ONE 2008; 3(6):e2341.
[4] Darling E M, Athanasiou K A. Rapid phenotypic changes in passaged articular chondrocyte subpopulations. J Orthop Res 2005; 23(2):425-32.
[5] Gilbert T W, Sellaro T L, Badylak S F. Decellularization of tissues and organs. Biomaterials 2006; 27(19):3675-83.
[6] Chen R N, Ho H O, Tsai Y T, Sheu M T. Process development of an acellular dermal matrix (ADM) for biomedical applications. Biomaterials 2004; 25(13):2679-86.
[7] Stapleton T W, Ingram J, Katta J, Knight R, Korossis S, Fisher J, et al. Development and characterization of an acellular porcine medial meniscus for use in tissue engineering. Tissue Eng Part A 2008; 14(4):505-18.
[8] Lumpkins S B, Pierre N, McFetridge P S. A mechanical evaluation of three decellularization methods in the design of a xenogeneic scaffold for tissue engineering the temporomandibular joint disc. Acta Biomater 2008; 4(4):808-16.
[9] Cartmell J S, Dunn M G. Effect of chemical treatments on tendon cellularity and mechanical properties. J Biomed Mater Res 2000; 49(1):134-40.
[10] Woods T, Gratzer P F. Effectiveness of three extraction techniques in the development of a decellularized bone-anterior cruciate ligament-bone graft. Biomaterials 2005; 26(35):7339-49.
[11] Liao J, Joyce E M, Sacks M S. Effects of decellularization on the mechanical and structural properties of the porcine aortic valve leaflet. Biomaterials 2008; 29(8):1065-74.
[12] Kasimir M T, Rieder E, Seebacher G, Silberhumer G, Wolner E, Weigel G, et al. Comparison of different decellularization procedures of porcine heart valves. Int J Artif Organs 2003; 26(5):421-7.
[13] Seebacher G, Grasl C, Stoiber M, Rieder E, Kasimir M T, Dunkler D, et al. Biomechanical properties of decellularized porcine pulmonary valve conduits. Artif Organs 2008; 32(1):28-35.
[14] Tudorache I, Cebotari S, Sturz G, Kirsch L, Hurschler C, Hilfiker A, et al. Tissue engineering of heart valves: biomechanical and morphological properties of decellularized heart valves. J Heart Valve Dis 2007; 16(5):567-73. Discussion 574.
[15] Grauss R W, Hazekamp M G, Oppenhuizen F, van Munsteren C J, Gittenberger-de Groot A C, DeRuiter M C. Histological evaluation of decellularised porcine aortic valves: matrix changes due to different decellularisation methods. Eur J Cardiothorac Surg 2005; 27(4):566-71.
[16] Meyer S R, Chiu B, Churchill T A, Zhu L, Lakey J R, Ross D B. Comparison of aortic valve allograft decellularization techniques in the rat. J Biomed Mater Res A 2006; 79(2):254-62.
[17] Meyer S R, Nagendran J, Desai L S, Rayat G R, Churchill T A, Anderson C C, et al. Decellularization reduces the immune response to aortic valve allografts in the rat. J Thorac Cardiovasc Surg 2005; 130(2):469-76.
[18] Rosario D J, Reilly G C, Ali Salah E, Glover M, Bullock A J, Macneil S. Decellularization and sterilization of porcine urinary bladder matrix for tissue engineering in the lower urinary tract. Regen Med 2008; 3(2): 145-56.
[19] Dahl S L, Koh J, Prabhakar V, Niklason L E. Decellularized native and engineered arterial scaffolds for transplantation. Cell Transplant 2003; 12(6): 659-66.
[20] Hodde J, Hiles M. Virus safety of a porcine-derived medical device: evaluation of a viral inactivation method. Biotechnol Bioeng 2002; 79(2):211-6.
[21] Hodde J, Janis A, Ernst D, Zopf D, Sherman D, Johnson C. Effects of sterilization on an extracellular matrix scaffold: part I. Composition and matrix architecture. J Mater Sci Mater Med 2007; 18(4):537-43.
[22] von Rechenberg B, Akens M K, Nadler D, Bittmann P, Zlinszky K, Kutter A, et al. Changes in subchondral bone in cartilage resurfacing—an experimental study in sheep using different types of osteochondral grafts. Osteoarthritis Cartilage 2003; 11(4):265-77.
[23] Elder B D, Kim D H, Athanasiou K A. Developing an articular cartilage decellularization process towards facet joint cartilage replacement. Neurosurgery, in press.
[24] Khalafi A, Schmid T M, Neu C, Reddi A H. Increased accumulation of superficial zone protein (SZP) in articular cartilage in response to bone morphogenetic protein-7 and growth factors. J Orthop Res 2007; 25(3): 293-303.
[25] Mauck R L, Nicoll S B, Seyhan S L, Ateshian G A, Hung C T. Synergistic action of growth factors and dynamic loading for articular cartilage tissue engineering. Tissue Eng 2003; 9(4):597-611.
[26] Saini S, Wick T M. Effect of low oxygen tension on tissue-engineered cartilage construct development in the concentric cylinder bioreactor. Tissue Eng 2004; 10(5-6):825-32.
[27] Elder B D, Athanasiou K A. Systematic assessment of growth factor treatment on biochemical and biomechanical properties of engineered articular cartilage constructs. Osteoarthritis Cartilage 2009; 17(1):114-23.
[28] Shimizu M, Minakuchi K, Kaji S, Koga J. Chondrocyte migration to fibronectin, type I collagen, and type II collagen. Cell Struct Funct 1997; 22(3):309-15.
[29] Rosenberg L. Chemical basis for the histological use of safranin O in the study of articular cartilage. J Bone Joint Surg Am 1971; 53:69-82.
[30] Brown A N, Kim B S, Alsberg E, Mooney D J. Combining chondrocytes and smooth muscle cells to engineer hybrid soft tissue constructs. Tissue Eng 2000; 6(4):297-305.
[31] Pietila K, Kantomaa T, Pirttiniemi P, Poikela A. Comparison of amounts and properties of collagen and proteoglycans in condylar, costal and nasal cartilages. Cells Tissues Organs 1999; 164(1):30-6.
[32] Woessner Jr J F. The determination of hydroxyproline in tissue and protein samples containing small proportions of this imino acid. Arch Biochem Biophys 1961; 93:440-7.
[33] Athanasiou K A, Agarwal A, Dzida F J. Comparative study of the intrinsic mechanical properties of the human acetabular and femoral head cartilage. J Orthop Res 1994; 12(3):340-9.
[34] Sneddon I. The relaxation between load and penetration in the axisymmetric Boussinesq problem for a punch of arbitrary profile. Int J Eng Sci 1965; 3: 47-57.
[35] Hayes W C, Keer L M, Herrmann G, Mockros L F. A mathematical analysis for indentation tests of articular cartilage. J Biomech 1972; 5(5):541-51.
[36] Athanasiou K A, Agarwal A, Muffoletto A, Dzida F J, Constantinides G, Clem M. Biomechanical properties of hip cartilage in experimental animal models. Clin Orthop Relat Res 1995; 316:254-66.
[37] Aufderheide A C, Athanasiou K A. Assessment of a bovine co-culture, scaffold-free method for growing meniscus-shaped constructs. Tissue Eng 2007; 13(9):2195-205.
[38] Elder B D, Athanasiou K A. Effects of temporal hydrostatic pressure on tissue engineered bovine articular cartilage constructs. Tissue Eng Part A, in press.
[39] Otzen D E. Protein unfolding in detergents: effect of micelle structure, ionic strength, pH, and temperature. Biophys J 2002; 83(4):2219-30.
[40] Gilbert T W, Freundb S J, Badylak S F. Quantification of DNA in biologic scaffold materials. Surg Res, in press.
[41] Revell C M, Athanasiou K A. Success rates and immunologic responses of autogenic, allogenic, and xenogenic treatments to repair articular cartilage defects. Tissue Eng Part B Rev 2009; 15(1):1-15.
[42] Platt J L, Fischel R J, Matas A J, Reif S A, Bolman R M, Bach F H. Immunopathology of hyperacute xenograft rejection in a swine-to-primate model. Transplantation 1991; 52(2):214-20.

What is claimed is:

1. A method for fabricating a tissue-engineered construct comprising: providing a tissue-engineered construct, wherein the tissue-engineered construct is derived from a xenogenic source; and decellularizing the tissue-engineered construct.

2. The method of claim 1 wherein tissue-engineered construct comprises chondrocytes.

3. The method of claim 1 wherein decellularizing the tissue-engineered construct comprises contacting the tissue-engineered construct with a compound chosen from one or more of a detergent, an organophosphorus compound, and a surfactant.

4. The method of claim 3 wherein decellularizing the tissue-engineered construct comprises contacting the tissue-engineered construct with a compound chosen from one or more of sodium dodecyl sulfate, tributyl phosphate, and polyethylene glycol p-(1,1,3,3-tetramethylbutyl)-phenyl ether.

5. The method of claim 3 wherein decellularizing the tissue-engineered construct comprises contacting the tissue-engineered construct with a compound chosen from one or more of about 1% sodium dodecyl sulfate, about 2% sodium dodecyl sulfate; about 2% tributyl phosphate, and about 2% polyethylene glycol p-(1,1,3,3-tetramethylbutyl)-phenyl ether.

6. The method of claim 3 wherein decellularizing the tissue-engineered construct further comprises contacting the tissue-engineered construct with one or more of a nuclease, a proteinase, an antibiotic, and an antifungal.

7. The method of claim 3 wherein decellularizing the tissue-engineered construct further comprises: introducing the tissue-engineered construct into a solution comprising phosphate buffered saline or culture media at 37° C.; and washing the tissue-engineered construct in the solution.

8. The method of claim 1 wherein providing a tissue-engineered construct comprises: providing a shaped hydrogel negative mold; seeding the mold with cells; allowing the cells to self-assemble in the mold to form a tissue engineered construct.

9. The method of claim 3 wherein the hydrogel is agarose or alignate.

10. The method of claim 3 wherein providing the shaped hydrogel negative mold comprises: coating at least one surface of a culture vessel with a molten hydrogel; inserting a shaped press into the molten hydrogel; allowing the molten hydrogel to cool around the press; and removing the press thereby leaving a shaped hydrogel negative mold.

11. The method of claim 1 wherein providing a tissue-engineered construct comprises: providing a shaped hydrogel negative mold and a shaped hydrogel positive mold; seeding the negative mold with cells; applying the positive mold to the negative mold; and allowing the cells to self-assemble within the negative and positive molds to form a tissue engineered construct.

12. The method of claim 1 wherein providing a tissue-engineered construct comprises: seeding cells in a hydrogel coated culture vessel; allowing the cells to self-assemble into a first construct; transferring the first construct to a shaped hydrogel negative mold; applying a shaped hydrogel positive mold to the negative mold to form a mold-construct assembly; and culturing the mold-construct assembly to form a second construct.

13. The method of claim 1 wherein providing a tissue-engineered construct comprises treating the tissue-engineered construct with a biochemical reagent, a mechanical force, hydrostatic pressure, or any combination thereof.

14. The method of claim 13 wherein the biochemical reagent is selected from the group consisting of a glycosaminoglycan depleting agent, a growth factor, chondroitinase-ABC, TGF-β1, and any combination thereof.

15. The method of claim 13 wherein the mechanical force is selected from the group consisting of direct compression, static hydrostatic pressure, non-static hydrostatic pressure, and any combination thereof.

16. The method of claim 1 wherein providing a tissue-engineered construct comprises coating at least one surface of a tissue culture vessel with a hydrogel; introducing onto the at least once hydrogel coated surface a suspension of live cells in culture medium; allowing the cells to sediment onto the coating to form an aggregate; and culturing the aggregate to yield a scaffoldless cartilage construct, or an intermediate thereof.

17. A tissue-engineered construct prepared by the method of claim 1 or claim 8.

18. A method for treating a subject comprising implanting in the subject a composition comprising at least one tissue engineered construct prepared by the method of claim 1 or claim 8.
